(19) United States
(12) Patent Application Publication
(10) Pub. No.: US 2013/0195840 A1
(43) Pub. Date: Aug. 1, 2013

(54) THERAPEUTICS AND PROCESSES FOR TREATMENT OF IMMUNE DISORDERS

(71) Applicants: Robert P. Kimberly, Birmingham, AL (US); Jeffrey C. Edberg, Birmington, AL (US); James M. Kelley, Opelika, AL (US)

(72) Inventors: Robert P. Kimberly, Birmingham, AL (US); Jeffrey C. Edberg, Birmington, AL (US); James M. Kelley, Opelika, AL (US)

(21) Appl. No.: 13/522,101

(22) PCT Filed: Jan. 13, 2011
(86) PCT No.: PCT/US11/21148

§ 371 Date: Oct. 15, 2012

Related U.S. Application Data
(60) Provisional application No. 61/335,862, filed on Jan. 13, 2010.

(51) Int. Cl. C12Q 1/68; G01N 33/68; A61K 31/675; A61K 31/519; A61K 39/395

(52) U.S. Cl. CPC C12Q 1/686; A61K 31/519; A61K 39/395; A61K 31/675; G01N 33/6854; G01N 33/6893
USPC 424/130.1; 514/262.1; 514/110; 435/6.11; 435/7.92; 435/7.21

(57) ABSTRACT

Processes of diagnosing or treating an autoimmune abnormality are provided whereby the presence of IgA anti-neutrophil cytoplasmic antibodies (ANCA) in a subject are detected correlating with both presence and severity of disease such as Wegener's granulomatosis (WG). The FCAR genotype predicts whether IgA ANCA will be stimulatory or inhibitory of neutrophil activation such that in subjects with an inhibitory genotype, IgA ANCA will act as an inhibitor of disease severity, and in subjects with a proinflammatory genotype, IgA ANCA will increase disease severity as observed by increased prevalence of renal disease in WG. Thus, individualized medical treatment is possible based on determination of the presence of IgA ANCA and FCAR genotype.

### CROSS REFERENCE TO RELATED APPLICATIONS

This application claims priority to U.S. Provisional Application No. 61/335,862 filed Jan. 13, 2010, the entire contents of which are incorporated herein by reference.

### GOVERNMENT SUPPORT

This invention was made with government support under Grant Nos. AR033062, AR049084, AR007450, AR042476, AR047799 and AR048095 awarded by the U.S. National Institutes of Health. The government has certain rights in the invention.

### FIELD OF THE INVENTION

The invention relates in general to autoimmune diseases or abnormalities. Processes and materials are presented that relate to cellular activation through the Fc family of receptors. Particularly, mechanisms related to Wegener's granulomatosis and the associated presence of IgA anti-neutrophil cytoplasmic antibodies and Fc receptor mediated regulation of neutrophil activation associated with presence or severity of disease are presented that are useful for diagnosis and screening of therapeutics for treating this rare, but severe disorder as well as other ANCA associated diseases.

### BACKGROUND OF THE INVENTION

Wegener's granulomatosis (WG) is a rare autoimmune condition marked by tissue damage to small and medium sized vessels as a result of aseptic inflammation. WG is almost exclusive to Caucasians (97.7%) with peak onset between ages 45 and 65 and occurs in approximately 1 out of 33,000 Caucasian individuals with no bias for gender.

A broad range of clinical manifestations are possible, but the American College of Rheumatology (ACR) diagnoses WG by the presence of at least two of the following clinical determinants: nasal or oral inflammation; abnormal chest radiograph; excessive urinary sediment; and granulomatous inflammation on biopsy. Alternatively, The Chapel Hill consensus criteria for systemic vasculitis places more emphasis on biopsy results.

Among the clinical manifestations, some form of renal involvement is reported in approximately 70-80% of WG patients. There is a range of renal involvement including decreased renal function (measured by elevated serum creatinine levels), decreased urine output, proteinuria, hematuria, and rapidly progressive glomerulonephritis. The severity of renal involvement is directly related to patient morbidity and mortality.

One area of investigation into the pathogenesis, severity, and prognosis of WG involves studying anti-neutrophil cytoplasmic antibodies (ANCAs). In WG, ANCAs typically recognize proteinase 3 (PR3, PRTN3), a serine protease primarily expressed in azurophilic granules of neutrophils. PR3-specific ANCAs (also known as cANCAs) fluorescently stain in a cytoplasmic pattern. Upon activation or priming, neutrophils can display PR3 on their cell surface with the majority of granule associated PR3 remaining membrane-associated. ANCAs bind to the surface of primed neutrophils, which express membrane associated ANCA targets such as PR3, resulting in initiation of neutrophil effector programs.

In the vasculitic neuropathies Churg-Strauss syndrome and microscopic polyangiitis, ANCAs recognize myeloperoxidase (MPO). MPO-specific ANCAs (p-ANCA) are characterized by their staining in a perinuclear pattern.

IgG ANCAs appear to be involved in the pathogenesis of vasculitic neuropathies such as WG and Churg-Strauss syndrome. While IgG is useful for understanding WG pathogenesis, the correlation of IgG titer to disease state is imperfect. Thus, there exists a need for processes and materials to diagnose or treat WG.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A process of diagnosing an autoimmune abnormality or predicting the severity of an autoimmune abnormality in a subject is provided including assaying a sample from a subject for the presence or absence IgA anti-neutrophil cytoplasmic antibodies (ANCA) to produce an IgA index, and diagnosing or predicting on the basis of the presence or absence of IgA ANCA in the sample. In genotypically unstratified populations as a whole, the identification of IgA ANCA in a blood, serum, or other biological sample is indicative of the presence of an autoimmune abnormality as well as suggests highly active disease. The inventive processes also optionally quantify the IgA ANCA in the sample as a way to predict whether IgA ANCA levels are increasing or decreasing over time or to predict disease remission or increased severity. The IgA index is optionally compared to a pre-determined an autoimmune abnormality classification system from a plurality of subjects of known autoimmune condition where a match to a parameter in the classification system allows diagnosing of the presence or susceptibility of autoimmune abnormality.

A genetic component to IgA ANCA mediated activity is observed. As such, an inventive process optionally includes determining the sequence of a polymorphic site in a FCAR gene of the subject and optionally from a second subject. The presence of a pro-inflammatory genotype, i.e. G allele at position 844 in FCAR, is indicative of a neutrophil stimulating effect of IgA ANCA whereas an A allele, which is present in the majority of the population, is indicative of an inhibitory IgA ANCA neutrophil effect. In these subjects, the presence of IgA ANCA correlates with reduced disease severity as observed by lower propensity for renal involvement in WG.

An inventive process optionally also includes assaying the sample for the presence or absence of IgG ANCA to produce an IgG index and optionally quantifies IgG ANCA present. Determining both IgG ANCA and IgA ANCA is indicative of disease or abnormality. The presence of renal involvement is optionally predicted based on the presence or absence of IgA ANCA and optionally the genotype of FCAR. The severity of an autoimmune abnormality is predicted from a positive IgA index, IgG index, or both. The autoimmune abnormality is optionally Wegener's granulomatosis.

Also provided is a process of treating an autoimmune abnormality in a subject including determining the sequence of a polymorphic site in a FCAR gene of a subject, and administering IgA to the subject. IgA therapy is most beneficial when a subject does not have a pro-inflammatory G allele at position 844 in FCAR. To avoid the possible complications from IgA therapy, a sample from a subject is optionally assayed for the presence of endogenous IgA, optionally prior to exogenous IgA administration.

As a treatment for autoimmune abnormality, an inventive process optionally includes administering to a subject with an autoimmune abnormality or having the propensity to develop an autoimmune abnormality, an inhibitor of CD89, optionally at a therapeutically effective amount. An inhibitor of CD89 optionally inhibits downstream signaling of CD89; CD89 engagement; or CD89 ligand binding. The process optionally also determines the sequence of a polymorphic site in a FCAR gene of the subject, optionally position 844, as a means to determine which type of therapeutic to administer. When a G is present at position 844 in FCAR, a therapeutic optionally inhibits CD89 engagement or CD89 ligand binding. When an A is present at position 844 in FCAR, a therapeutic optionally modulates CD89 mediated signaling.

An inventive process optionally also includes determining whether one or more copies of the PRTN3 gene comprise a pre-determined polymorphic sequence.

A process of screening for a therapeutic or selecting a therapeutic to treat a disease or autoimmune abnormality is also provided including contacting a potential therapeutic with a cell, and identifying increased or decreased IgA-ANCA induced activity in the cell where IgA-ANCA induced activity is any alteration in cellular activity, structure, or composition, illustratively degranulation, expression extracellular protein, or NET formation.

A process of selecting a therapeutic to treat a disease or autoimmune abnormality in a subject is also provided including determining whether one or more copies of the FCAR gene of the subject has an A or G allele at position 844, and selecting a therapeutic based on the result. The A allele indicates IgA based therapies may be desired to increase CD89 engagement, and a G allele indicates that CD89 signaling based therapies may be desired.

Any of the inventive processes also optionally includes determining whether said subject has a NA1 or NA2 allele of FCGR3B. Any of the inventive processes optionally also includes administering a CD89 inhibitor or activator to a subject.

Processes are also provided for predicting the severity of an autoimmune abnormality in a subject including determining whether one or more copies of the FCAR gene of the subject has an A or G allele at position 844, ascertaining whether the subject has a NA1 or NA2 allele of FGCR3B, and predicting the severity of an autoimmune abnormality in a subject on the basis of the determining and the ascertaining. The process optionally includes assaying a sample from the subject for the presence or absence of IgA anti-neutrophil cytoplasmic antibodies to produce an IgA index, and diagnosing or predicting also on the basis of the presence or absence of the antibodies in the sample.

### BRIEF DESCRIPTION OF THE DRAWINGS

FIG. 1 is a plot illustrating the increased prevalence of IgA ANCA in patients with more active WG; and

FIG. 2 depicts experimental results illustrating human IgG and IgA anti-PR3 containing antibody fractions impact degranulation in neutrophils from healthy donors (n=4) measured as change in CD11b surface expression by flow cytometry with differences in degranulation compared to stimulation with IgG and IgA antibody fractions not containing anti-PR3;

FIG. 3 depicts NET formation results stratified by FCAR genotype (rs16986050), where the GG genotype, which results in a more pro-inflammatory response, is associated with higher percentage of cells with NET formation when stimulated with IgA anti-PR3 antibody containing serum (p=0.008);

FIG. 4 depicts experimental results stratified by FCGR3B NA1/NA2 genotype, where the more pro-inflammatory NA1 genotype results in a higher percentage of cells with NET formation when stimulated by IgG ANCAs (p=0.03); and

FIG. 5 depicts experimental results of neutrophils stimulated with IgA anti-PR3 antibody containing serum can reduce the NET formation potential of IgG ANCA stimulation.

### DETAILED DESCRIPTION OF EMBODIMENTS OF THE INVENTION

The following description of embodiments of the invention is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention, but are presented for illustrative and descriptive purposes only. It is further appreciated that the individual steps of the processes taught herein are interchangeable, and one of ordinary skill in the art recognizes beneficial ways of making such interchangments to the elements of the inventive processes. It is further appreciated that the inventive processes are operable to select a subject for treatment or treatment type, to select a therapeutic to be administered to a subject, determine the degree of responsiveness a subject has or is likely to have from administration of a therapeutic, select a subject for participation in a clinical study, determine or predict abnormality severity, determine or select the therapeutic regimen to use to best treat a subject, or for other uses readily appreciated by one of ordinary skill in the art or as illustrated herein.

The inventors unexpectedly discovered that IgA anti-neutrophil cytoplasmic antibodies (ANCAs) are present in patients presenting with WG and that these levels correlate with disease activity. As used herein the term IgA ANCA is intended to mean immunoglobulin A species directed to protein endogenous to a subject that produces the immunoglobulin A. Illustratively, ANCA antibodies bind PR3 such as the PR3 present on neutrophils or myeloperoxidase (MPO) such as ANCAs associated with Churg-Strauss syndrome. As such, when the term IgA anti-PR3 is used it is appreciated that these represent a subset of IgA ANCA. Similarly, when the term IgG anti-PR3 is used that this represents as subset of IgG ANCA.

The inventors also identified a genetic association between small nucleotide polymorphisms (SNPs) in FCAR and disease state indicating that the IgA fraction of ANCAs are functional determinants of WG. The invention has utility as a diagnostic for ANCA related diseases, for the determination or prediction of the severity of ANCA related diseases, in the development of therapeutics to vasculitis, or for developing individualized therapy to vasculitis.

The inventive identification of IgA ANCAs from patients with WG reveals new mechanisms of disease pathogenesis the regulation of which will promote treatment. Similarly, the identification of CD89-mediated activities in WG provides new avenues of therapeutic development. The presence of IgG ANCAs produces signaling through the FcγII and FcγIII receptors. In addition to signaling via these receptors, IgA ANCAs introduce signaling via CD89 (FcαR), the magnitude and type of which will affect the severity of disease or propensity to develop disease. Upon binding PR3, IgA ANCAs engage signaling through CD89 via the Fc portion of the antibody.

The inventors also discovered a genetic component to the function of IgA ANCAs that dramatically and unexpectedly affects the severity of disease. In subjects with a pro-inflammatory CD89 sequence (Gly²⁴⁸) resulting from a G allele at position 844 in FCAR, activation of CD89 promotes IL-6 cytokine release, intracellular calcium mobilization, and degranulation in neutrophils. The heightened immune cell activity leads to increased propensity for disease, increased disease severity, or reduction in propensity for remission. In these subjects the presence of IgA ANCAs increases disease severity by signaling for additional cellular activation via CD89.

In subjects with the more common CD89 sequence (Ser²⁴⁸) resulting from the A allele at position 844 in FCAR, engagement of CD89 produces an inhibitory signal that correlates with a reduction in disease severity as observed by reduced renal involvement. In these subjects the presence of IgA ANCAs is beneficial because engagement of CD89 reduces cell activation as measured by relative reduced IL-6 release, lower changes in CD-11b expression, and reduced NET formation. Overall, while the presence of IgA ANCAs alone correlates with the presence of autoimmune disease and disease severity in the genetically unstratified population as a whole such that the detection of IgA ANCAs alone is sufficient to diagnose disease, the specific activities of the IgA ANCAs are related to their level in the bloodstream and the subject's CD89 sequence.

Although the invention is described herein with relation to WG, the invention is not intended to be limited as such. One of skill in the art will readily understand other diseases or abnormalities that are similarly diagnosable or treatable by the invention.

An inventive process is provided for diagnosing the presence or absence of an autoimmune abnormality in a subject. A first process illustratively includes assaying a sample from a subject for the presence or absence of IgA ANCAs in the sample to obtain an IgA index. A sample is illustratively a first sample, a second sample, a third sample, or a fourth sample. An IgA index is the outcome of an assay that positively or negatively identifies the presence of IgA ANCA when used in this context. An IgA component affect correlating to autoimmune disorders such as WG is considered in the prior art as unimportant due to the levels of IgG ANCAs from patients with WG. The inventors surprisingly discovered that subjects with WG present IgA ANCAs. Further, the level of IgA ANCAs correlates with WG severity.

A subset of WG forms or severities are illustratively affected by the presence of IgA ANCAs. The presence of IgA ANCAs correlates with mucocutaneous manifestations such as nasal or oral inflammation and cutaneous vasculitis. As used herein, “severity” with respect to WG is the presence or absence of renal involvement with subjects with renal involvement demonstrating more severe forms of WG and subjects without renal involvement or less life threatening renal involvement demonstrating less severe forms of WG.

An inventive processes illustratively include obtaining a sample from a subject and assaying the sample for the presence of IgA ANCA. A sample that assays positive for IgA ANCA correlates with the presence of autoimmune abnormality or susceptibility to autoimmune abnormality. The absence of detectable IgA ANCA in a sample correlates with a diagnosis of no autoimmune abnormality or low susceptibility to autoimmune abnormality.

Table 1 depicts the association of IgA ANCAs with the presence of autoimmune abnormality as detected in two population studies of subjects presenting with WG. The WGGER group includes samples from 502 subjects. The VCRC group includes samples from 374 subjects. Overall, subjects with WG are much more likely to have IgA ANCAs in the bloodstream than are subjects without WG.

TABLE 1

| | Controls | WGGER | VCRC |
|---|---|---|---|
| IgG Positive | 1 (1%) | 121 (48.4%) | 136 (51.7%) |
| IgA Positive | 1 (1%) | 50 (20%) | 101 (38.4%) |
| IgA and IgG Positive | 0 (0%) | 39 (15.6%) | 56 (21.3%) |
| Negative | 97 (98%) | 118 (47.2%) | 82 (31.2%) |
| Total | 99 | 250 | 263 |

Thus, an inventive process uses the presence of IgA ANCAs as a basis for diagnosing the presence or absence of an autoimmune abnormality.

It is appreciated that the presence or absence of other characteristics of disease are optionally used in making a diagnosis. Illustratively, nasal or oral inflammation, abnormal chest radiograph, excessive urinary sediment, or granulomatous inflammation on biopsy are used to confirm or lend support to a diagnosis based on the presence or absence of IgA ANCAs.

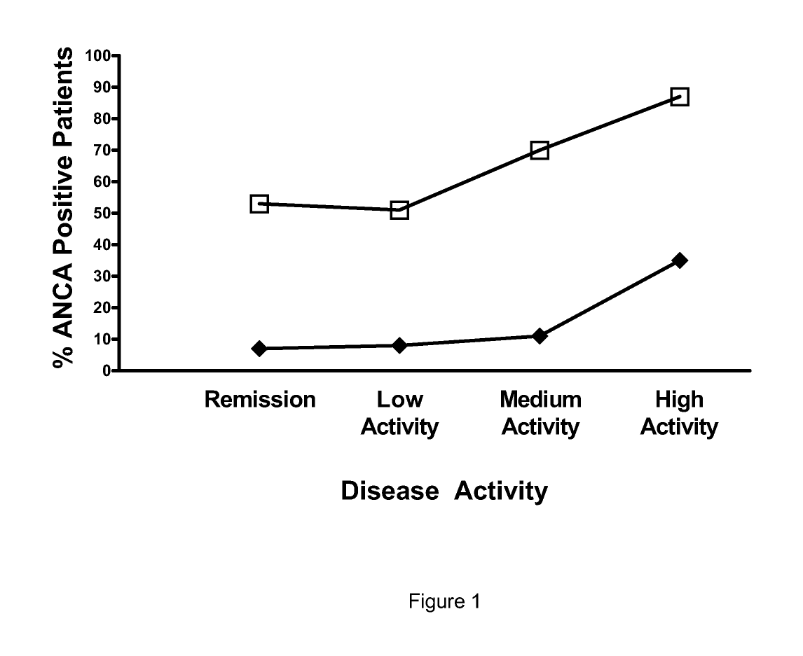

The presence of autoimmune abnormality is optionally predicted or diagnosed based on the level or presence of IgA ANCAs in a sample obtained from a subject. As seen in FIG. 1, subjects with IgG and IgA ANCAs are more likely to have highly active disease. Thus, the presence of IgA ANCAs is expected to differ dependent on the stage or activity of disease and can be used as a marker of disease remission or reintroduction. FIG. 1 illustrates that subjects in remission or lower activity WG are less likely to have detectable IgA ANCAs. A subject with IgG ANCAs but no IgA ANCAs are less likely to have highly active WG. Finally, subjects with both IgG and IgA ANCAs are more likely to have highly active WG.

As used herein, a “subject” refers to a cell or organism. Organisms illustratively include: humans; non-human primates illustratively including monkey, chimpanzee, and others; horses; goats; cows; sheep; pigs; dogs; cats; guinea pigs; hamsters; rabbits; mice; rats; or other rodents. A subject is illustratively a patient suffering a disease or condition.

As used herein, the term “autoimmune abnormality” is a disease or condition resulting from or correlating to autoimmunity. An autoimmune abnormality is optionally related to the activation state of immune cells, illustratively neutrophils, or other polymorphonuclear cells whether immune related or not. An autoimmune abnormality is optionally related to CD89 mediated activities illustratively including phagocytosis, antibody-dependent cell-mediated cytotoxicity (ADCC), and cytokine production, among other activities known in the art. Illustratively, a disease is optionally related to platelet activation or platelet activity. A disease is optionally related to monocyte activation as ANCAs are also known to lead to monocyte chemotaxis by triggering MCP-1 secretion. A disease or abnormality is illustratively: a systemic vasculitide illustratively including Churg Strauss syndrome (CSS), polyarteritis nodosa, microscopic polyangiitis, and Wegener's granulomatosis (WG); rheumatoid arthritis (RA); systemic lupus erythematosus (SLE); lupus nephritis; Kawasaki disease; immune glomerulonephritis; inflammatory bowel disease (e.g., Crohn's disease); immune diabetes; and other immunoregulated diseases including IgA nephropathy, Sjögren's syndrome, celiac disease, immune thrombocytopenia (ITP), thrombosis, myocardial infarction, anticardiolipin syndrome, and stroke.

As used herein, the term “sample” is a biological sample. A biological sample illustratively includes whole blood, plasma, serum, extracellular fluid, cytosolic fluid, isolated or unisolated cells, tissue, solubilized cellular membrane samples, cultured cells, cell culture media, and physiological buffered forms thereof.

An “assay” is any process operable for detecting or evaluating a biological or chemical composition, illustratively the presence of IgA ANCA in a sample. An assay is illustratively an enzyme linked immunosorbent assay (ELISA), chromatography, mass spectrometry, affinity assay, nucleic acid sequencing including single nucleotide sequence determination, protein sequencing, determinant of cell activation, flow cytometry, electrophoresis, or other assay known in the art. An assay is optionally suitable to quantify the level of IgA ANCAs in a sample. Illustratively, an ELISA can quantify the level of IgA ANCAs by comparison to a standard curve. As such, an inventive process optionally includes quantifying the amount of IgA ANCAs, IgG ANCAs, or both in a sample from a subject.

The presence or quantity of IgA ANCAs, IgG ANCAs, or both are optionally determined at more than one time in one or more samples from a subject. In some embodiments, a first sample is obtained from a subject and the presence, absence, or amount of IgA or IgG ANCAs is determined in the sample at a first time. A second sample is optionally obtained at a time before, simultaneous with, or after a first sample is obtained and the presence, absence, or amount of IgA or IgG ANCAs is determined in the second sample at a second time. A third, fourth, fifth, or additional sample is optionally obtained at a time before, simultaneous with, or after a first sample and the presence, absence, or amount of IgA or IgG ANCAs is determined in the second sample at a corresponding time. As such, a temporal trend of IgA ANCA characteristics are obtained. This trend may be used to predict or determine disease progress, remission, or reintroduction.

An inventive process illustratively includes creating an autoimmune classification system. An autoimmune classification system is a datum or data from one or more subjects with a known disease or autoimmune abnormality state, or with a known response to a therapeutic. Illustratively, an autoimmune classification system includes a datum or data about the IgA or IgG ANCA content in a sample from one or more subjects with no known disease or autoimmune abnormality. Illustratively, an autoimmune classification system is a datum or data about the IgA, IgG, or both, ANCA content in a sample from one or more subjects with a known disease or autoimmune abnormality. Optionally an autoimmune classification system is data from subjects with and subjects without a disease or autoimmune abnormality.

Illustratively, an autoimmune classification system includes data from a plurality of subjects with known disease state or known autoimmune condition. An autoimmune classification system is illustratively presented in Table 2. Thus, by comparing the presence or absence of IgA ANCA from a subject of unknown disease state, it is possible to diagnose WG or predict WG severity. If a subject has IgA ANCAs, IgG ANCAs, or both, comparing this information as an IgA index, and IgG index, or both to an autoimmune classification system can be used to diagnose disease.

TABLE 2

| | WG Subjects | Controls |
|---|---|---|
| IgG ANCA+ | 121 (48.4%) | 1 (1%) |
| IgA ANCA+ | 50 (25.0%) | 1 (1%) |
| IgG/IgA ANCA+ | 39 (15.6%) | 0 (0%) |
| ANCA Negative | 118 (47.2%) | 97 (98%) |
| Total | 250 | 99 |

An autoimmune classification system is optionally constructed from results obtained from samples from a plurality of subjects at one or more times.

An inventive process illustratively includes determining whether one or more copies of a FCAR gene in a subject have one or more pre-determined polymorphic sequences or SNPs. The FCAR gene encodes the CD89 protein. The inventors surprisingly discovered the pre-determined polymorphic sequences of the FCAR gene correlate with the presence of disease, including WG. Polymorphic sequences are illustratively found in U.S. Pat. No. 6,986,987, and WO 00/05403, the contents of each of which are incorporated herein by reference for the whole of their teaching including how to detect the sequence of FCAR from one or more genes in a subject.

Illustratively, the pre-determined polymorphic sequence in FCAR is denoted rs16986050 (844A →G corresponding to amino acid change S248G), rs61735070 (836C →T corresponding to amino acid change P245L), rs11666735 (376G→A corresponding to amino acid change D92N), rs1865096 (variant at position chr19:55396900 is a coding region synonymous polymorphism of A (Arg108) to G (Arg) [where the A/G is the nucleotide change and Arg is the corresponding amino acid change]), or rs12975083 (variant is at position chr19:55398235 and is an intronic polymorphism of C679→T). Other FCAR sites of polymorphic sequences include rs2304225, rs3816051, rs4806608, and rs7259347.

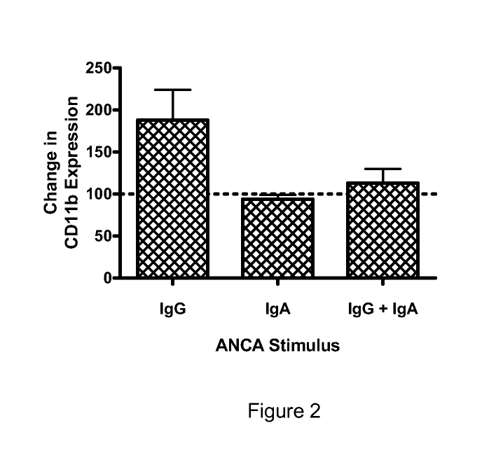

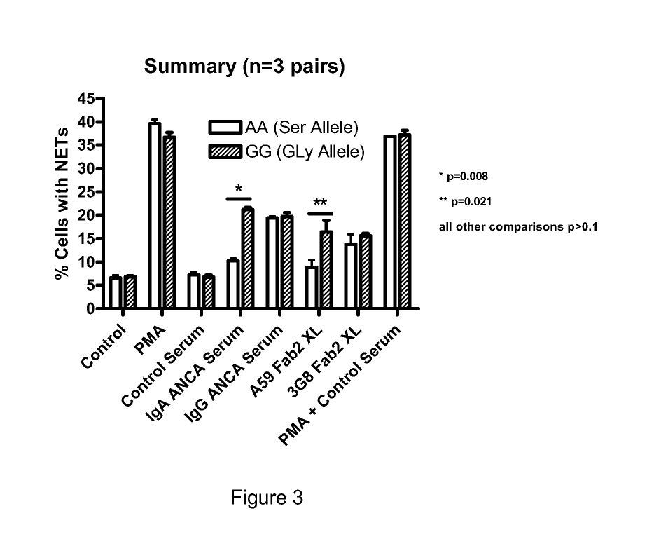

In some embodiments, a FCAR gene in a subject has an adenine (A) at position 844 that correlates to a CD89 protein with a Ser at position 248. The presence of A at 844 produces a CD89 protein that functions as a neutrophil activation inhibitor. Without being bound by one particular theory, the Ser²⁴⁸ in CD89 is capable of being phosphorylated. The phosphorylated Ser²⁴⁸ is capable of interacting with intracellular signaling proteins that result in an inhibitory activity toward neutrophil activation as demonstrated by suppression of neutrophil chemotaxis, down-regulation of the generation of reactive oxygen species, and the induction of the anti-inflammatory cytokine IL-1ra. In addition, the inventors demonstrate that in populations with the more common SNP of A at position 844 in FCAR that IgA ANCAs are unable to activate neutrophils alone and actually inhibit IgG ANCA mediated neutrophil activation (FIGS. 2 and 3) As such, some embodiments of the invention include determining the sequence of a polymorphic site in a FCAR gene and from this determining diagnose autoimmune abnormality or predict the severity of autoimmune abnormality from the determining optionally combined with an index as to the presence or absence of IgA ANCAs in a sample obtained from a subject. When a subject has both the A844 allele and IgA ANCA's the severity of disease or propensity of disease development is reduced.

In some embodiments, a FCAR gene has a guanine (G) at position 844 that correlates with a Gly at amino acid position 248 in the CD89 protein. A Gly is unable to be phosphorylated, thus, altering the signaling by a CD89 protein. A CD89 protein with Gly²⁴⁸ is pro-inflammatory. (Wu, et al., J. Immunol., 2007; 178: 3973-3982). Without being limited to one particular theory, the Gly²⁴⁸ CD89 protein is capable of binding Lyn independent of interactions with an FcγR protein, and is thereby capable of inducing activating intracellular signaling events upon binding an IgA ligand. The inventors demonstrate that the ability of CD89 mediated neutrophil activation by anti-PR3 containing IgA antibody fractions (IgA ANCAs) or the anti-PR3 monoclonal antibody CLB12.8 to activate neutrophils as measured by NET formation is dependent on whether CD89 contains a Gly at position 248. (FIG. 3). Little to no NET formation is observed in neutrophils derived from AA homozygous subjects, but robust NET formation is observed from neutrophils from GG homozygous subjects. As such, some embodiments include determining the presence or absence of an activating G allele at position 844 in FCAR and from this determining combined with an index as to the presence or absence of IgA ANCAs in a sample obtained from the subject, diagnose an autoimmune abnormality or predict the severity of an autoimmune abnormality. A subject with a G allele at position 844 in FCAR that demonstrates IgA ANCAs is more likely to have autoimmune abnormalities with a greater disease severity.

As depicted in Table 3, subjects from two combined WG groups show a significant increase in the severity of the disease as measured by the presence or absence of renal involvement when a G allele is present. The G allele is found in subjects in 10.9% of subjects without any renal manifestation compared to 18.9% of subjects with renal disease (P=0.02).

TABLE 3

| | Genotype | | | Allele | |
|---|---|---|---|---|---|
| Cases | AA | AG | GG | A | G |
| No Renal | 232 | 51 | 6 | 515 | 63 |
| involvement | 80.3% | 17.6% | 2.7% | 89.1% | 10.9% |
| (WGGER and VCRC) | | | | | |
| Renal | 226 | 111 | 10 | 563 | 131 |
| Involvement | 65.1% | 32.0% | 2.9% | 81.1% | 18.9% |
| (WGGER and VCRC) | | | | | |
| All Cases | 455 | 164 | 16 | 1074 | 196 |
| (WGGER and VCRC) | 71.7% | 25.8% | 2.5% | 84.6% | 14.4% |

As such, the presence of a G allele alone correlates with disease severity. When subjects are further stratified by the presence or absence of IgA ANCAs it is expected that combined IgA ANCAs and a G allele at 844 in FCAR correlates highly with renal involvement in WG.

The sequence of a polymorphic site in the FCAR gene, the FCGR3B gene, or both is appreciated to be achieved by one or more of several techniques known in the art including direct gene sequencing, sequencing of mRNA or cDNA, protein sequencing (i.e. amino acid indicates codon sequence in gene), Taqman or like protocols, mass spectrometry, immune related techniques illustratively ELISA, and other protocols known in the art. As such, the term “determining” when related to determining a sequence is used herein as an assay or protocol by any technique known to those of skill in the art for the determination of the sequence of at least a portion of a gene or a protein.

The determined sequence is optionally compared to a sequence in a FCAR gene from a second or plurality of other subjects that are either control subjects without known autoimmune abnormalities or with a known autoimmune abnormality. The comparing of the determined sequence and the known sequence supports diagnosis of autoimmune abnormality or predicts severity of autoimmune abnormality.

An inventive process optionally includes determining whether one or more copies of the PRTN3 gene has a polymorphic sequence. The PRTN3 gene encodes the target for ANCAs, the proteinase 3 protein (PR3), a serine protease primarily expressed in azurophilic granules of neutrophils. A polymorphic sequence of PRTN3 is optionally rs351111 which is a variant at position chr19:795020 producing a missense polymorphism of A→G (Val→Ile at position 118) where the A/G is the nucleotide change and the Val/Ile is the corresponding amino acid change.

Also provided is a process of treating a disease or autoimmune abnormality including administering a therapeutic to a subject. As used herein the term “therapeutic” refers any molecule or therapy that affects or is effected by immune cells or immune mediators. A therapeutic optionally blocks the binding of IgA ANCA binding to PR3 or an Fc receptor such as CD89. A therapeutic is optionally an inhibitor of CD89. An inhibitor of CD89 optionally inhibits direct or downstream signaling of CD89, prevents CD89 engagement by preventing CD89 oligomerization or translation of signal from an extracellular ligand binding site to an intracellular signaling motif, or blocks CD89 ligand binding. A therapeutic is optionally an inhibitor of one or more FcγRs that may function by inhibiting direct or downstream signaling of a FcγR, prevents FcγR engagement by preventing FcγR oligomerization or translation of signal from an extracellular ligand binding site to an intracellular signaling motif, or blocks FcγR ligand binding. Illustratively, a therapeutic is selective for a particular FcγR or FcγR allele or is a pan inhibitor that will effectively inhibit signaling by several types of FcγR.

Optionally a therapeutic is an immunosuppressive agent. Illustrative examples of immunosuppressive agents include cyclophosphamide, rilonacept, sirolimus, mycophenolic acid, mycophenolate mofetil, cyclosporine, tacrolimus, methotrexate, and azathioprine. Illustratively, a therapeutic is: a corticosteroid, illustratively, prednisone, methyl prednisone, or Medrol; an antibiotic, illustratively, trimethoprim/sulfamethoxazole; plasmapheresis; radiation exposure; an immunoglobulin; an immunogen; an antigen; cytokines; interleukins; ES-62; and any chemotherapeutic listed in Strome, S E, et al, The Oncologist, 2007; 12:1084-1095, the entire contents of which are incorporated herein by reference.

A therapeutic is optionally an immunoglobulin (Ig) or immunoglobulin fragment. An immunoglobulin is optionally polyclonal or monoclonal, an immunoglobulin fragment, or a fusion protein. An immunoglobulin is optionally a fully human monoclonal antibody, a murine monoclonal antibody, a chimeric monoclonal antibody, or a humanized monoclonal antibody. An immunoglobulin is illustratively: antibody 2B6; antibody CC49; anti-FcαRI Fab; anti-FcαRI MAb; IgA complex; antibody conjugates; rituximab; infliximab; abciximab; trastuzumab; canakinumab; cetuximab; alemtuzumab; omalizumab; efalizumab; or abatacept. It is appreciated that an immunoglobulin is optionally modified to be tolerated by a subject. Optionally, an immunoglobulin is humanized by processes known in the art. An immunoglobulin is optionally derived from plasma and is termed “plasma derived.” Immunoglobulins derived from plasma are optionally isolated from one or more other components normally present in the plasma from a donor organism. Optionally, an immunoglobulin is a portion of an immunoglobulin fraction optionally derived from plasma. In some embodiments plasma derived immunoglobulin is isolated so as to be free, or substantially free of other plasma components.

An immunoglobulin is illustratively an IgG, IgA, IgE, IgD, IgM or combinations thereof. An immunoglobulin is optionally an immunoglobulin fragment, illustratively a Fab domain. Optionally, an immunoglobulin is IgA. Traditional immunoglobulin therapy is performed with IgA depleted immunoglobulins. The invention unexpectedly identifies a use for exogenous IgA in that administration of IgA can compete with IgA ANCAs as well as reduce the severity of disease, eliminate disease, or put disease in remission. Methods of immunoglobulin therapy are known in the art. Determining the levels of IgA administered to a subject is performed by known methods. These methods are regularly practiced in the art. As used herein, the term exogenous is defined as derived from a source other than the subject to which a therapeutic is to be administered. Immunoglobulin derived from plasma, serum, or whole blood, chemically synthesized, or recombinantly expressed are illustrative examples of exogenous immunoglobulins or therapeutics.

An IgA is optionally capable of binding a PR3 and is, thus, an IgA ANCA. Exogenous IgA ANCAs are particularly useful when administered to a subject without an activating CD89 sequence. The exogenous IgA ANCA will both compete with endogenous IgG ANCAs for PR3 binding sites as well as stimulate the inhibitory CD89 sequence preventing neutrophil activation and reducing disease severity. An exogenous immunoglobulin, including an exogenous IgA ANCA is optionally a sequence that is modified to bind more or less tightly to PR3 than an endogenous immunoglobulin. An exogenous immunoglobulin may be able to more effectively compete with endogenous immunoglobulin, require lower dosages or frequency of dosages, and may be able to stimulate CD89 more effectively.

The administration of IgA, as an example, is of particular importance in a host with activating or inhibiting polymorphic sequences in CD89. Illustratively, the nonsynonymous SNP in the coding region of CD89 (844A→G) (rs16986050), which changes codon 248 from AGC (Ser²⁴⁸) to GGC (Gly²⁴⁸) in the cytoplasmic domain of CD89 demonstrates significantly different signaling activity. Wu, J., et al., J. Immunol., 2007; 178:3973-3982. Screening a subject for the presence or absence of one or more alleles provides valuable insight into which subjects will most benefit from IgA administration. A subject that expresses the rs16986050 G allele shows increased CD89-mediated IL-6 cytokine release by human neutrophils and thus will benefit from a therapeutic that inhibits IgA ANCA interactions with CD89 or IgA ANCA-induced CD89 engagement.

The inventive process optionally includes administering IgG or a fragment thereof to a subject. IgG is optionally co-administered with IgA. Administration of IgG is illustratively prior to, coincident with, or following IgA administration. The co-administration of IgG and IgA provides beneficial clinical outcome for diseases such as WG.

Therapeutic administration is illustratively performed on a daily, weekly, monthly, or annual basis. It is appreciated that a person of skill in the art will adjust the administration schedule according to the needs of the subject. A subject's needs are illustratively an additional dose, an increased dose, a missed dose, or other physiological requirement recognized by one of skill in the art.

A therapeutic is optionally selected based on the determined genotype of a subject or from assays to determine the presence or absence IgA ANCA mediated disease. Illustratively, a therapeutic that blocks engagement of CD89 via the IgA Fc domain is more likely to benefit a subject with a G allele at position 844 of FCAR and may not benefit a subject with two A alleles. Similarly, a subject with the a G allele at position 844 of FCAR demonstrates increased neutrophil IL-6 production and will benefit from IL-6 targeted therapy. IL-6 increases serum concentrations of acute phase proteins, reduces the level of serum albumin, and affects the presence of remarkable thrombocytosis. Moreover, IL-6 is capable of stimulating IL-6 receptor (IL-6R) negative cells such as vascular endothelial cells when complexed to soluble form of IL-6R (sIL-6R). As such, a subject presenting WG with increased IL-6 production due to the a G allele at position 844 of FCAR will be more likely to benefit from treatment with atlizumab or another anti-IL-6 antibody such as that described by Ito, H., Curr Pharm Des. 2003; 9(4):295-305. Similarly, degranulation resulting from IgA ANCA mediated CD89 activation is modulatable by TGF-β1, or nordihydroguaiaretic acid and the lipoxygenase/cyclo-oxygenase inhibitor, 5,8,11,14-eicosatetraynoic acid.

In some embodiments, a therapeutic targets Bruton's tyrosine kinase (Btk). Btk is a cytoplasmic member of the Tec family of kinases and is important in B-lymphocyte development, differentiation, and signaling. The inventors discovered that signaling by CD89 (Gly²⁴⁸) passes through an activation of Btk. It is also possible that some level of signaling by CD89 (Ser²⁴⁸) also proceeds through Btk activation, but is not identical. Thus, by administering an inhibitor to a downstream signaling molecule such as Btk, the severity of autoimmune abnormality can be reduced, particularly in subjects expressing CD89 (Gly²⁴⁸). Illustrative examples of Btk inhibitors include PCI-32765, AVL-292, AVL-101, PCI-32765, LFM-A13, or other Btk inhibitor known in the art. It is similarly appreciated that administration of any therapeutic that inhibits downstream signaling through the pro-inflammatory CD89 (Gly²⁴⁸) may be particularly beneficial in subjects that also express IgA ANCAs. As such, assaying for the presence or absence of IgA ANCAs or alleles that correlate with disease severity allow selection of a therapeutic to treat the disease.

The level of therapeutic and frequency and type of administration are readily envisioned and determined by one of ordinary skill in the art. Illustratively, a therapeutic is administered orally or parenterally depending on the oral bioavailability and tolerability of the therapeutic. Administration is optionally intravenously, intrathecally, subcutaneously, injection, or by other method known in the art.

It is appreciated that more than one therapeutic may be administered to a subject to treat an auto immune disorder. Other therapeutics illustratively include cyclophosphamide, methotrexate alone or in combination with glucocorticoids, glucocorticoid monotherapy or combined therapy with methotrexate or cyclophosphamide, azathioprine, trimethoprim-sulfamethoxazole, intravenous immunoglobulin, antithymocyte globulin, plasmapheresis or plasma exchange, tumor necrosis factor (TNF) antagonists illustratively etanercept or infliximab, rituximab, leflunomide, mycophenolate mofetil (MMF), 15-deoxyspergualin (DSG), or other therapeutics or therapies known in the art. Therapeutically effective doses, frequency of dosing, and considerations for therapeutic or therapy type are illustratively found in Wung, P K and Stone, J H, Nat Clin Prac Rheum, 2006; 2:192-200, and the references cited therein, each of which are incorporated herein by reference in their entirety.

The frequency of therapeutic administration is illustratively daily, weekly, monthly, or several times a day or every set period of hours. The dosing frequency is readily determined by one of ordinary skill in the art with readily obtained or already understood knowledge of the pharmacokinetics of the therapeutic. Illustratively, PCI-32765 is administered orally once a day at a therapeutically effective amount of between 1.25 mg/kg/day to 17.5 mg/kg/day, optionally at 560 mg daily. IgA is optionally administered once daily at a dose of 1-2 gm/kg. IgA administration is illustratively continued for four to five days. It is appreciated that in some embodiments more than one therapeutic is optionally administered simultaneously.

A therapeutic is optionally administered in a therapeutically effective amount. A “therapeutically effective amount” is any amount sufficient to induce a response sufficient to prevent, reduce, alter, or ameliorate signs or symptoms of an autoimmune abnormality and thereby treat a disease or condition. Signs or symptoms of an autoimmune abnormality illustratively include: activation of any polymorphonuclear cell such as neutrophils, basophils, eosinophils, megakaryocytes, or platelets; presence of increased levels of IL-8, IL-6, CD-11b expression, NET formation, or other signs of polymorphonuclear cell activity; dolor; calor; rubor; tumor; fever; weight loss; palpable purpura; livedo reticularis; myalgia; arthralgia; mononeuritis multiplex; headache; stroke; tinnitus; reduced visual acuity; acute visual loss; nose bleeds; bloody cough; lung infiltrates; abdominal pain; bloody stool; GI perforations; alteration in erythrocyte sedimentation rate; elevated C-reactive protein; hematuria; glomerulonephritis; elevated serum creatine; presence of ANCA; abnormal biopsy including the presence of granulomatous inflammation; abnormal chest radiograph; abnormal angiogram; or other sign or symptom known in the art. A sign or symptom is ameliorated if any desired change in the level of the sign or symptom is observable.

As IgA is depleted from immunoglobulin therapy in traditional administrations because of a desire to avoid immunizing subjects with an IgA deficiency and potentially causing anaphylactic shock, the inventive process optionally includes screening a subject for the presence or absence of an IgA deficiency. Methods of screening for IgA deficiency are known in the art. Optionally, IgA deficiency is determined by latex agglutination inhibition such as the process described by Munks, R., Transfusion Science, 2004; 10:155-59. Optionally, an IgA ELISA technique is used such as that described by Kramer, J., et al., Haematologia (Budap), 1988; 21(4):233-8. It is appreciated that other methods known in the art are similarly operable. The presence or absence of IgA deficiency is optionally determined in a subject prior to administering a therapeutic.

Also provided is a process of screening for a therapeutic suitable to treat a disease or condition, such as an autoimmune disease or condition including contacting a chemical or biological compound that is a potential therapeutic with a cell and identifying increased, decreased, or no change in affector induced activity in the cell. An affector is illustratively any composition that will alter a characteristic of a cell, tissue, or organism. An affector illustratively alters the activation state of a cell, changes the level, activity, or activation state of one or more intracellular, membrane, transmembrane, or cell surface proteins, nucleic acids, lipids, fatty acids, ions, or other chemical component of a cell. Other chemical, physical, or other alteration in a cell is equally envisioned as occurring as a result of contact with an affector.

An affector is illustratively an immunoglobulin affector such as an IgA or IgG. Illustrative examples are isolated or modified immunoglobulins. An immunoglobulin affector is optionally an IgA ANCA or an IgG ANCA. It is appreciated that multiple affectors may be used simultaneously or sequentially to produce a desired activity in a cell, tissue, or organism.

A potential therapeutic is any chemical or biological composition that has known or unknown activity toward another molecule. Illustratively, a chemical or biological agent that is the subject of a screen or other test to determine its activity or its use as a control of the determination of another compound's activity is a potential therapeutic. The term “potential therapeutic” also encompasses compositions with known therapeutic efficacy.

An inventive process also includes selecting a therapeutic to treat a disease or abnormality such as an autoimmune abnormality. Treating a disease or abnormality is altering any sign or symptom of disease. An inventive process illustratively includes determining whether one or more copies of the FCAR gene of a subject has an A or G allele at position 844, and selecting a therapeutic based on the determined FCAR allelic composition of the subject. As elsewhere referred to herein, the presence of an A allele will produce a CD89 protein that is inhibitory of neutrophil activity and is best subject of a therapeutic that will increase receptor engagement. The presence of a G allele will produce an activating CD89 protein and is best subject to an inhibitor of CD89 mediated signaling. It is appreciated that in some embodiments overlapping signaling mechanisms could be subject to a therapeutic that will be efficacious in treating a disease or autoimmune abnormality independent of whether a subject has the G or A allele in FCAR.

A process optionally includes assaying a sample from a subject for the presence or absence of IgA anti-neutrophil cytoplasmic antibodies to produce an IgA index and selecting a therapeutic on the basis of determining the FCAR allele and the IgA index. Illustratively, a subject without IgA ANCA might benefit from administration of intravenous IgA, particularly exogenous IgA ANCA, when the subject has the A allele of FCAR. Alternatively, a subject with the G allele of FCAR and with the presence of IgA ANCA may benefit from an inhibitor of CD89 mediated signaling whereas a subject with the G allele and without IgA ANCA may find less benefit from such a signaling inhibitor.

The role of Fcγ receptors in autoimmune disorders may also contribute to the selection of a therapeutic. IgG ANCAs are direct pathogenic effectors in WG via their interaction with FCγRs. Genetic variants of neutrophil-expressed FCγRs also have an important role in disease. The genetic polymorphisms in FCGR2A and FCGR3A are associated with disease relapses in WG (Dijstelbloem, et al, Arthritis Rheum, 1999; 42 1823-7), and copy number variation of FCGR3B is associated with development of systemic autoimmune conditions including WG (Fanciulli et al, Nat Genet, 2007; 39:721-3). Expression of an arginine from the G allele of the FCGR2A H131R polymorphism (rs1801274) reduces binding affinity for IgG2 and IgG3 while increasing recognition of C-reactive protein.

There are two common genetic variants of FCGR3B, named NA1 and NA2, with the NA1 allele producing a stronger phagocytic, respiratory burst, and degranulation response compared to the NA2 allele. The NA1 allele of FCGR3B is related to increased neutrophil surface PR3 expression, which supports a role for genetic variants such as FCGR3B-NA1 not only influencing disease susceptibility but impacting WG severity as well. As such an inventive process optionally includes determining whether a subject has a NA1 or NA2 allele of FCGR3B. A subject with the NA1 allele may be more receptive to anti-IgG ANCA activity mediated therapy or to treatment with exogenous IgA ANCA due to the competition for PR3 reducing stimulation of the FCγRs. Similarly, a subject with the NA1 allele may be a better candidate for FCγR signaling inhibitors.

It is appreciated that the inventive processes are not limited to the NA1 or NA2 alleles of FCGR3B. Other allelic variants that alter the expression, signaling, or other activity of FCGR3B are similarly operable in the invention.

A classification system optionally is a 3×3 matrix incorporating and relating disease severity to FCAR genotype and FCGR3B genotype. An illustrative matrix is illustrated in Table 4 where the greater the disease activity or severity is indicated with more (+) symbols than lower disease activity of severity.

TABLE 4

FCAR (A/G 844)

| Gene | Allele | A/A | A/G | G/G |
|---|---|---|---|---|
| FCGR3B | NA1/NA1 | ++ | ++++ | ++++ |
| | NA1/NA2 | + | +++ | ++++ |
| | NA2/NA2 | + | ++ | ++ |

As such, some embodiments of the inventive processes include determining the allelic sequence of the A/G 844 polymorphism in FCAR and the presence of the NA1/NA2 allele of FCGR3B. This genetic information is compiled from a plurality of subjects with known disease and disease severity to form the classification system of Table 4. Each cell in the exemplary classification system of Table 4 represents a classification category including information related to abnormality severity, abnormality activity, or propensity to develop an abnormality from one or more subject's with known autoimmune condition (i.e. normal or abnormality). It is appreciated that while the classification system of Table 4 is a two-dimensional 3×3 matrix, that additional information may be added or removed from the classification system. Optionally, a classification system includes information related to the presence or absence of IgA ANCA, IgG ANCA, or both. As such a three dimensional or other classification system is developed and used to select a subject for treatment or treatment type, to select a therapeutic to be administered to a subject, determine the degree of responsiveness a subject has or is likely to have from administration of a therapeutic, select a subject for participation in a clinical study, determine or predict abnormality severity, determine or select the therapeutic regimen to use to best treat a subject, or for other uses readily appreciated by one of ordinary skill in the art or as illustrated herein.

As an exemplary use of a classification system, a subject presents with possible autoimmune disorder based on the display of one or more traditional symptoms. A sample from the subject is used to determine the sequence of polymorphic sites in FCAR and FCGR3B and the results compared to the classification system to determine not only whether the subject has the propensity to develop a disease or abnormality, but also how severe the disease or abnormality is or is likely to become. In this example, the subject is homozygous G/G at position 844 in FCAR and NA1/NA1 homozygous in FCGR3B. These results are compared to the classification system which indicates that subjects with both these genotypes have severe abnormality such as the occurrence of renal involvement. The treating physician then begins aggressive treatment so as to counteract current or future complications from the abnormality.

The classification system of Table 4 also optionally is used to determine the type, frequency, or dose of therapeutic to administer to a subject. Illustratively, a subject that has or is likely to develop highly active WG or WG with renal involvement due to being homozygous G/G at position 844 in FCAR and NA1/NA1 homozygous in FCGR3B, will be given more aggressive therapy than a subject that is homozygous G/G at position 844 but also NA1/NA1 homozygous in FCGR3B which indicates less severe WG. As an illustration, a patient is genotyped homozygous A/A at position 844 in FCAR and NA1/NA1 homozygous in FCGR3B. This subject is likely to have moderately active WG or mild renal involvement based on the classification system of Table 4. This is due to the homozygous A/A at position 844 in FCAR reducing disease severity combating with the NA1/NA1 homozygous in FCGR3B that produces more severe disease. This subject is then, as an illustration, given plasma derived IgA therapy of moderate intensity, optionally IgA ANCA therapy, and also treated with IgG-Fc region binding peptide TG19320, or other FcγR activity inhibitor to both maximize the inhibitory activity of the CD89 and reduce the activity of the FcγR.

In some embodiments, a classification system includes information about the presence or absence of IgA ANCA. This information can be in addition to the information captured in Table 4 to form a three dimensional matrix that provides more information to a physician making treatment decisions or an investigator determining subject to include or exclude from a clinical trial.

One of ordinary skill in the art recognizes how to both create and interpret a classification system based on genotyping as well as how to compare a subject's genotype to a classification system to determine how aggressively to treat and what therapeutic(s) to administer to treat or prevent an autoimmune abnormality.

Various aspects of the present invention are illustrated by the following non-limiting examples. The examples are for illustrative purposes and are not a limitation on any practice of the present invention. It will be understood that variations and modifications can be made without departing from the spirit and scope of the invention. A person of ordinary skill in the art readily understands where reagents for the practice of the invention may be obtained.

### Example 1

Determination of Fc receptor alleles: To determine the alleles of FCAR, genomic DNA is extracted from leukocytes (in EDTA anti-coagulated whole blood) using with the Puregene DNA isolation kit (Gentra Systems, Minneapolis, Minn.). Allele-specific PCR assays are used to genotype donors for the FcαRI alleles illustratively including: FcαRIa (ECI)-87R/87R, FcαRIA (ECI)-92D/92N, FcαRIa (EC2)-132F/132L, FaRI (CY)-245P/245L and FcαRI (CY)-248S/248G alleles. Since there is a finite error rate in any genotyping assay, each assay is corroborated by direct dye-primer based cycle sequencing of at least 40 homozygous donors and an equal number of heterozygous donors. In addition, each assay includes blinded but known genotyped controls to verify the fidelity of the assay. Ambiguities in the assay are resolved by first repeating the allele-specific PCR reaction and then by direct dye-primer based cycle sequencing of genomic DNA samples.

Alternatively, an oligonucleotide ligation assay (OLA) is used. In OLA the polymorphic residue is within the PCR amplicon. Three oligonucleotides are then added: one that is immediately 3′ to the polymorphic residue; a fluorescently labeled oligonucleotide that is complementary to one allele at the 3′ end; and a fluorescently labeled third oligonucleotide that is complementary to the other allele at the 3′ end. The two labeled primers are of different lengths (different by >2 nucleotides) and can be labeled with the same or different fluorescent probes (such as 6-FAM and tet for detection on the ABI377). This technique is now widely used for analysis of mutations in the cystic fibrosis gene as described by Tokoro Y, et al., J Oral Pathol Med, 1996; 25:225-231.

FCGR genotyping is performed by isolation of genomic DNA extracted from leukocytes (in EDTA anti-coagulated whole blood) using the Puregene DNA isolation kit (Gentra Systems, Minneapolis Minn.) and assays to determine the genotype of FcγRIIIB and FcγRIIIA In brief, allele-specific PCR reactions are used and along with direct sequencing of gene specific amplicons to determine the FcγRIIIb-NA1 and NA2 alleles and the FcγRIIIa-176F and 176V alleles. Sequencing is performed on an ABI 377 (ABI, Foster City, Calif.). The PCR products are gel purified with the QIAquick Gel Extraction Kit prior to sequencing (Chatsworth, Calif.).

### Example 2

Purification of Immunoglobulins (Igs): Human serum Igs are purified by chromatography using anion exchange (Mono Q), molecular sieve (Superose 6 or 12, or Sephacryl S-300), affinity (jacalin-HiTrap for IgA1; protein G-HiTrap for IgG), and immunoadsorbent (anti-IgM-HiTrap) columns in an FPLC apparatus (Pharmacia Biotech, Piscataway, N.J.) or conventional columns. IgA subclasses are separated by means of a jacalin-HiTrap column which retains IgA1 while IgA2 passes; IgA1 is then recovered by elution with 0.1 M melibiose. Monomeric and polymeric IgA are separated by HPLC on a Biosep Sec-53000 column (Phenomenex, Torrance, Calif.), or for larger quantities by FPLC on a Sephacryl S-300 Hi-Prep column. All these procedures are capable of achieving>99% purity of Ig isotypes. Purity and concentration are assessed by SDS-PAGE and ELISA.

### Example 3

Assay of immunoglobulins (Ig) by ELISA: Determination of the presence of IgA ANCA is performed using a modified version of the INOVA Diagnostics (San Diego, Calif.) QUANTA Lite™ PR-3 ELISA kit (catalog #708705) and the MPO ELISA kit (catalog #708700). These ELISA kits quantify the level of IgG anti-PR3 or IgG anti-MPO autoantibodies in human serum. The assay methodology for IgA is followed exactly as recommended by the manufacturer except that an anti-IgA-HRP (INOVA Diagnostics, catalog #508549, HRP IgA Conjugate (goat), anti-human IgA) is used in place of the anti-IgG-HRP conjugate to determine the level of IgA ANCA in the samples. As a positive control for IgA, the positive control reagents contained in the QUANTA Lite Gliadin ELISA (INOVA Diagnostics. Catalog #704525) are used following the manufacturers recommended procedure. As standards, samples from 90 healthy controls are used to define a positive IgA titer value that is greater than the average OD450 of all controls plus 2 standard deviations, as is standard practice. A 98% agreement is found between our method and the manufacturer's method to define positivity.

To further characterize Igs, ELISA plates are coated with anti-Ig antibodies of the desired specificity (Dako Corp., Carpinteria, Calif.), at optimal levels (1-10 μg/ml). The plates are blocked with 0.15% Tween-20 in PBS, and serially diluted samples of purified immunoglobulins of Example 2, starting from a dilution appropriate to the sample and the expected analyte concentration, are applied overnight. Bound Ig is detected by means of peroxidase-conjugated antibody to the analyte, diluted to the previously determined optimal level in each case (usually 1:1,000-1:5,000), and applied for 4 h. When IgA subclasses are assayed, the bound Ig is detected with monoclonal anti-IgA1 or anti-IgA2 antibodies (Nordic Immunological Labs., Capistrano Beach, Calif.), followed by peroxidase-conjugated anti-mouse Ig (Southern Biotechnology, Inc., Birmingham, Ala.). Finally, the color developed with a substrate of o-phenylenediamine (0.5 mg/ml) plus 1 mM H₂O₂ after 15 min is read at 490 nm in an ELISA plate reader (MRX; Dynatech Laboratories, Chantilly, Va.) interfaced to a computer for data retrieval and processing. Controls include the use of uncoated (blocked) wells, and coated wells treated with all reagents but not exposed to analyte sample. All determinations are performed with at least duplicate samples. Where appropriate, the assay is calibrated by a serially diluted standard (Human Ig Reference Preparation; The Binding Site, San Diego, Calif.), and a standard curve is generated by computer program based on the 4-parameter logistic method.

### Example 4

IgA ANCA is identified in WG subjects and their presence correlates with disease severity. Plasma samples obtained from subjects from two studies are used to determine the presence or absence of IgA ANCA and correlate IgA ANCA to disease severity. The Wegener's Granulomatosis Genetics Repository (WGGER), is a cross-sectionally assembled collection of 502 patients and 413 healthy controls enrolled at multiple centers across the United States including Beth Israel Medical Center (New York, N.Y.), Boston University (Boston, Mass.), the Cleveland Clinic Foundation (Cleveland, Ohio), Duke University (Durham, N.C.), John Hopkins University (Baltimore, Md.), the Lahey Clinic (Burlington, Mass.), the Mayo Clinic (Rochester, Minn.), and the University of Alabama at Birmingham (Birmingham, Ala.) [coordinating center] with approval of each respective Institutional Review Board and individual patient informed consent. Due to the difficulty in recruiting patients with a disease that has a prevalence of 1 per 33,000, WGGER serves as the largest single collection of patients with WG and controls assembled to date.

Among all patients enrolled, 463 (93%) are Caucasian, and 48% are male, which are similar demographics for disease to those reported in the literature. Ethnicity was self-defined and confirmed by multi-dimensional principal component analysis using ancestry informative markers derived from 768 single nucleotide polymorphisms (SNPs) genotyped previously on an Illumina GoldenGate Genotyping BeadXpress system. The average age for onset of symptoms in WGGER is 47.1 years with ages ranging from 13 to 86 (median=48).

Expert medical chart review confirmed the presence of at least two of the four diagnostic criteria established by the American College of Rheumatology (ACR) for Wegener's Granulomatosis (WG) in 477 patients, which were used in further analyses. Clinical histories detailing demographics, ACR diagnostic criteria, organ system involvement, ANCA status, and disease severity were submitted by referring physicians and stored in a Microsoft Access database. 58.5% of patients had some form of renal involvement, which included hematuria, elevated serum creatinine, or end stage renal disease. The mean peak serum creatinine recorded was 3.07 mg/dL (range: 0.7-22.0 mg/dL; median: 1.7 mg/dL). Given that the average time from diagnosis to enrollment in the cross-sectional study was 5.48 years, it is likely that any progression to severe renal disease or development of other manifestations would have occurred. Furthermore, no significant difference in the age or disease duration was found between patients with or without renal involvement, thereby lowering the possibility of any lead time bias.

The Vasculitis Clinical Research Consortium (VCRC) is a multi-centered research infrastructure based in multiple North American academic medical centers, including Boston University (Boston, Mass.) [coordinating center], the Cleveland Clinic (Cleveland, Ohio), Johns Hopkins University (Baltimore, Md.), the Mayo Clinic (Rochester, Minn.), McMaster University (Hamilton, ON, Canada), and the University of Toronto (Toronto, ON, Canada). The VCRC collects longitudinal comprehensive clinical data and linked biological samples from patients with giant cell arteritis, Takayasu's arteritis, polyarteritis nodosa, microscopic polyangiitis (MPA), Churg-Strauss syndrome (CSS), and Wegener's granulomatosis (WG). All participants were recruited with informed consent under supervision of Institutional Review Boards and met classification criteria established by the ACR and/or the Chapel Hill Consensus definitions of disease for vasculitis. In the current study, only patients with WG, MPA, and CSS were included. At the time of this study, there were 1,470 biological samples collected at various time points from 263 patients with WG, 79 samples from 15 patients with MPA, and 485 samples from 96 patients with CSS.

Subjects with WG averaged 5.6 visits (range 1-19, median −4) with visit intervals most frequently 3 months but ranging from 1 month to 1 year. Due to overlapping recruitment centers, 67 patients with WG were enrolled in both WGGER and VCRC and are excluded from analyses where appropriate.

IgA ANCA is detected in 29.4% of patients tested from both WGGER and VCRC as measured using ELISA as described in Example 3 and overall depicted in Table 1. The presence of IgA ANCA is confirmed using indirect immunofluorescence assays (IFA) with neutrophil substrate from the NOVA Lit^{e}™ ANCA (INOVA Diagnostics) and read on a NIKON fluorescence microscope. Toat anti-human anti-IgG conjugated with FITC (INOVA) and goat anti-human anti-IgA conjugated with Texas Red (Southern Biotech, Birmingham, Ala.) are used as secondary antibodies. Similar to IgG ANCAs, IgA ANCAs primarily target PR3, and titers vary over time. Capture ELISAs using recombinant PR3 as antigen detect IgA anti-PR3 antibodies in 11 of 35 WG patients (31.4%), replicating a similar prevalence with independent samples and techniques.

The level of WG activity correlates with the presence of IgA ANCA. As seen in FIG. 1, subjects with highly active WG show IgA ANCA in approximately 40% of cases. The prevalence of IgA ANCA decreases with decreasing severity. These data roughly correlate with increased prevalence of IgG ANCA. (FIG. 1).

The presence of IgA ANCA also correlates with the presence of renal disease such as end-stage renal disease (ESRD). Within VCRC, end-stage renal disease (ESRD) occurs in 20.8% of IgA anti-PR3-negative patients compared to 4.9% of IgA anti-PR3-positive individuals (P=0.006). Within WGGER, 11.5% of patients develop ESRD compared to 4.5% of IgA anti-PR3-positive patients; mucosal upper airway inflammation occurred in 98.0% of IgA anti-PR3-positive patients compared to 81.9% of IgA anti-PR3-negative patients (P=0.004). Therefore, the presence of IgA ANCA antibodies is less frequently observed in individuals with severe renal disease and more common in patients presenting with mucosal upper airway manifestations.

Samples from the subjects in VCRC are also examined for the presence of anti-MPO IgA ANCAs. Among patients with CSS, another disease marked by upper airway abnormalities and kidney involvement, IgA anti-MPO antibodies are more prevalent than IgG anti-MPO antibodies (50% vs. 29.2%, P=3.1×10⁻³) (Table 5), and, analogous to observations in WG, IgA anti-MPO-positive patients are less likely to require renal dialysis compared to IgA anti-MPO-negative individuals [6/48 (12.7%) vs. 9/47 (19.2%)]. These data indicate that the presence of anti-MPO IgA ANCAs correlates with a reduction in disease severity in CSS similar to the correlation between the presence of anti-PR3 IgA ANCAs and a reduced likelihood of renal involvement in WG.

TABLE 5

| | Controls | CSS |
|---|---|---|
| IgG Positive | 1 (1%) | 28 (29.2%) |
| IgA Positive | 0 (0%) | 48 (50%) |
| IgA and IgG Positive | 0 (0%) | 12 (12.5%) |
| Negative | 89 (99%) | 32 (33.3%) |
| Total | 90 | 96 |

### Example 5

The presence of the pro-inflammatory G allele in FCAR correlates with increased WG severity. Genotyping of haplotype tagging single nucleotide polymorphisms (htSNPs) spanning FCAR from samples from both the WGGER and VCRC is performed using Illumina GoldenGate Genotyping BeadXpress system (San Diego, Calif.) following all manufacturer protocols on 463 Caucasian patients and 413 Caucasian controls from WGGER and using Pyrosequencing (QIAGEN, Hilden, Germany) for 261 Caucasian patients from VCRC. htSNPs are selected using pairwise comparisons with the Tagger algorithm, which is part of HaploView v3.31. The r² threshold is set at 0.8 and limited genotype data to SNPs with minor allele frequencies (MAF)>0.05 in the HapMap Caucasian (CEU) population (Phase II/Release 20). Genotyped SNPs include: rs11666735, rs12975083, rs16986050, rs1865096, rs2304225, rs3816051, rs4806608, and rs7259347. The SNPs (rs11666735) is not compatible with the Illumina platform so that this SNP is genotyped with a TaqMan allelic discrimination assay (Applied Biosystems [ABI], Foster City, Calif.) in 5 μL volume reactions using an ABI7900HT. All SNPs are in Hardy-Weinberg Equilibrium.

As seen in Table 6, the ‘G’ allele of rs16986050 (844 A to G), which generates an increased inflammatory response to IgA, is found in 10.9% of WG patients without any renal manifestation compared to 18.9% patients with renal disease (P=0.01). There are also significantly different allele frequencies between the mucosal (upper airway involvement) (15.4%) and nonmucosal (21.6%) patient groups (P=0.02).

TABLE 6

| | Genotype | | | Allele | |
|---|---|---|---|---|---|
| Cases | AA | AG | GG | A | G |
| No Renal | 149 | 38 | 3 | 336 | 44 |
| Involvement | 78.4% | 20% | 1.6% | 88.4% | 11.6% |
| (WGGER) | | | | | |
| Renal | 171 | 77 | 7 | 419 | 91 |
| Involvement | 67.1% | 30.2% | 2.7% | 82.1% | 17.8% |
| (WGGER) | | | | | |
| All Cases | 320 | 115 | 10 | 755 | 135 |
| (WGGER) | 71.9% | 25.8% | 2.2% | 84.8% | 15.2% |
| No Renal | 83 | 13 | 3 | 179 | 19 |
| Involvement | 83.8% | 13.1% | 3.0% | 90.4% | 9.6% |
| (VCRC) | | | | | |
| Renal | 55 | 34 | 3 | 144 | 40 |
| Involvement | 59.8% | 37.0% | 3.3% | 78.3% | 21.7% |
| (VCRC) | | | | | |
| All Cases | 135 | 49 | 6 | 319 | 61 |
| (VCRC) | 71.1% | 25.8% | 3.2% | 83.9% | 16.1% |
| No Renal | 232 | 51 | 6 | 515 | 63 |
| Involvement | 80.3% | 17.6% | 2.7% | 89.1% | 10.9% |
| (WGGER and VCRC) | | | | | |
| Renal | 226 | 111 | 10 | 563 | 131 |
| Involvement | 65.1% | 32.0% | 2.9% | 81.1% | 18.9% |
| (WGGER and VCRC) | | | | | |
| All Cases | 455 | 164 | 16 | 1074 | 198 |
| (WGGER and VCRC) | 71.7% | 25.8% | 2.5% | 84.6% | 14.4% |
| Controls | 249 | 149 | 15 | 647 | 179 |
| (WGGER) | 60.3% | 36.1% | 3.6% | 78.3% | 21.7% |
| HapMap | 75 | 32 | 6 | 182 | 44 |
| Caucasians | 66.4% | 28.3% | 5.3% | 80.5% | 19.5% |

Assays to determine FCGR3B genotype in patients from both the WGGER and VCRC groups are performed as described in Example 1. The NA1 allele is related to severe renal disease in WG: 26% of NA1 homozygote positive patients develop ESRD compared to 11.5% among those not homozygous for NA1. The average peak serum creatinine level is also higher among the NA1 homozygotes (4.51 mg/dL) compared to all (renal and non-renal) patients (3.07 mg/dL) or even to non-NA1 patients with renal disease (3.67 mg/dL). Homozygosity for the less activating allele, NA2, is present in 38% of patients with mucosal upper airway manifestations compared to 30% of patients without this type of mucosal involvement.

No difference is observed in copy number variation between patients and controls.

### Example 6

IgA and IgG ANCA function differently as neutrophil activation stimuli in general population studies. For all neutrophil studies, washed whole blood is used to avoid isolation induced neutrophil activation. Briefly, blood obtained by venipuncture into the anti-coagulant EDTA is chilled to 4° C., washed twice in modified phosphate buffered saline (PBS) (125 mM sodium chloride, 10 mM phosphate, 5 mM potassium chloride, 5 mM glucose, pH 7.35), and then resuspended in the original volume.

In experiments to measure neutrophil degranulation (CD11b surface expression), washed cells are pre-incubated for 40 minutes at 37° C. to induce cell surface expression of PR3. Aliquots are then incubated with or without anti-PR3 mAb CLB 12.8 (10 mg/ml) or anti-PR3 containing antibody fractions for 45 min. Anti-CD11b monoclonal antibodies (mAb) are obtained from Caltag (San Francisco, Calif.) and anti-PR3 mAb CLB 12.8 (mIgG1) from Research Diagnostics (Flanders, N.J.). Human anti-PR3 containing antibody fractions (IgA and IgG) are jacalin and protein G sepharose column purified from five patients with known high titers; each is further reversely depleted to avoid contamination. Isolation is confirmed by SDS-PAGE and western blot using Fcα- and Fcγ-specific antibodies (Jackson ImmunoResearch Laboratories Inc.) and confirmed reactivity with anti-PR3 ELISA. Endotoxin levels in all reagents are below detection limits by the limulus ameobocyte assay (Sigma, St. Louis, Mo.). After stimulation, samples are treated with FACS Lysing Solution (Becton Dickinson Immunocytometry, San Jose, Calif.) for 10 minutes at room temperature, washed once, and analyzed by flow cytometry (FACSCaliber, Becton Dickinson). Neutrophils are identified by characteristic light-scatter properties and FCγR staining with mAbs IV.3 and 3G8 (Medarex Inc., Annandale, N.J.). Cell surface expression of PR3 displays a bimodal pattern. Analysis of flow cytometry data is performed using CellQuest (Becton Dickinson). The results are expressed as mean fluorescence intensity (MFI) of the histogram data.

The neutrophils from healthy donors are stimulated with column-purified IgA and/or IgG antibody fractions from patients with high anti-PR3 titers and from ANCA-negative controls. Measurements of degranulation reveal a substantial CD11b increase compared to control IgG. Stimulation with the IgA anti-PR3 containing antibody fraction, however, produce virtually no change in CD11b compared to control IgA (FIG. 2). IgA ANCA in neutrophils from these donors is inhibitory of degranulation by IgG ANCA as is seen in the nearly 40% decrease in degranulation when neutrophils are activated in the presence of both IgA and IgG ANCAs relative to IgG ANCA alone (P=0.031). Thus, in donors representing the majority of the population, IgG ANCA mediated degranulation of neutrophils is inhibited by the presence of IgA ANCA.

The above inhibitory affects of IgA ANCA above observed using neutrophils from donors with the more common yet less inflammatory FCAR allele with A at position 844. To identify whether different FCAR alleles alter the results, the above assays are repeated using donors with the pro-inflammatory variant (G at position 844). Neutrophils with the active allele demonstrate that IgA anti-PR3 antibodies can increase neutrophil degranulation, albeit in an allele specific manner. IgG ANCA also induces neutrophil activation in a FcR-dependent manner, as observed by a dose dependent reduction in CD11b expression in the presence of IgG:FcR engagement inhibiting peptide TG19320 or soluble IgG FcRs (FCGR2A/FCGR2B), adding evidence that ANCA-induced neutrophil stimulation is FcR related.

Another measure of neutrophil activation is the formation of neutrophil extracellular traps (NETs) that are believed to promote renal inflammation in small vessel vasculitis. To measure NETs, neutrophils from FCAR genotyped donors (as in Example 5) are primed with tumor necrosis factor (TNF) and stimulated with IgA and IgG antibody fractions from healthy controls or from patients with known high anti-PR3 ELISA titers (either IgA or IgG). After 180 minutes, NET formation is visualized using Hoechst 33342 staining by fluorescent microscopy using pseudocolor to enhance contrast. Phorbol myrisitate acetate (PMA) is used as a positive control stimulus.

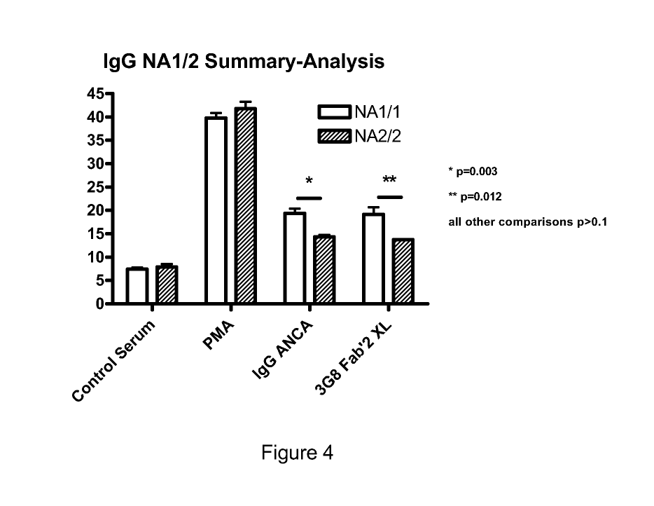

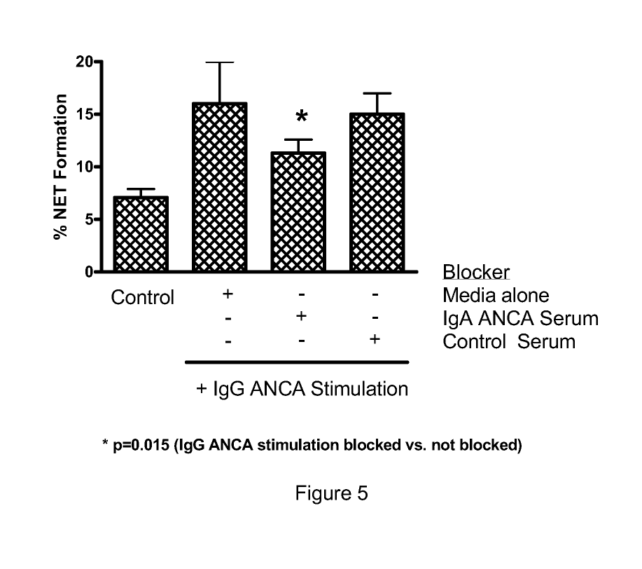

Enhanced NET formation is observed using neutrophils obtained from individuals with the pro-inflammatory G allele. (FIG. 3) Also, stimulation with IgG anti-PR3 containing serum increases NET formation in NA1-homozygotes more than NA2-homozygous individuals. (FIG. 4). When neutrophils are stimulated with IgA and IgG anti-PR3 containing fractions simultaneously, a reduction in NET formation (P=0.015) is observed (FIG. 5).

### Examples 7 and 8

Diagnosing and treating unknown subjects. Five patients present several symptoms including fatigue, weight loss, fever, shortness of breath, bloody sputum, joint pain, sinus inflammation (sinusitis), nasal ulceration, or bloody nasal discharge. Each of the five patients is tested for the presence or absence of the traditional WG clinical determinants nasal or oral inflammation, abnormal chest radiograph, excessive urinary sediment, or granulomatous inflammation on biopsy. Patients are also tested or observed for traditional CSS clinical determinants of: allergic reactions including asthma, hay fever or sinusitis; hypereosinophilia; or vasculitis. All patients demonstrating at least one traditional clinical determinant are tested for the non-traditional clinical determinants including blood testing for the presence of IgG ANCA and IgA ANCA, and genetic screening for FGCR3B alleles and FCAR alleles using procedures in Examples 1-3. Three of the five patients present with IgG ANCA, IgA ANCA, both, or at least two of the traditional WG clinical determinants and are diagnosed with WG.

The three positive WG patients are further screened for FCAR alleles. Table 7 illustrates the results of the genotype determination of FCAR alleles A/G at position 844 as well as the presence or absence of IgA ANCA.

TABLE 7
Characteristics of WG patients.

| Patient | FCAR genotype | IgA ANCA | IgA |
|---|---|---|---|
| A | AA | (+) | (+) |
| B | AA | (−) | (−) |
| C | GG | (+) | N/D |

A (+) indicates a positive result; A (−) indicates a negative result.

The remaining two patients are positive for CSS as measured by the presence of IgG ANCA, IgA ANCA, or both as well as at least one traditional CSS determinant. These patients are also screened for FCAR alleles and correlate to patients A and C in Table 7 such that similar treatments are administered.

Treatment decisions are made based on the determined IgA ANCA content and the determined FCAR genotype. Patient A is homozygous AA indicative of an inhibitory function to CD89 as well as the presence of IgA ANCA. The IgA ANCAs alone are indicative of the presence of WG. Patient B is also homozygous AA, but does not show the presence of IgA ANCA. Patients A and B are, therefore, good candidates for a therapeutic that will increase the activation of CD89 such as intravenous IgA therapy. This is particularly relevant for subject B who does not present with IgA ANCA to act as an inhibitory agent to neutrophil activation, which explains the presence of renal involvement to their WG, a complication not observed in patient A. Prior to intravenous IgA, both patient A and B are screened for the presence of endogenous IgA so as to prevent unwanted complications from therapy. Patient A has endogenous IgA but patient B does not. Therefore, patient A is administered intravenous IgA as a therapeutic to treat their WG. Patient B is given a traditional therapy of cyclophosphamide dosed at 2 mg/kg/day.

Patient C is homozygous GG at position 844 in FCAR. A CD89 engaging therapy such as intravenous IgA is inappropriate for this patient. Patient C is administered the Btk inhibitor PCI-32765 orally at a dose of 560 mg/day.

All WG and CSS patients show improvement in following therapeutic administration by reduced severity or remission.

Various modifications of the present invention, in addition to those shown and described herein, will be apparent to those skilled in the art of the above description. Such modifications are also intended to fall within the scope of the appended claims.

Methods involving conventional biological techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; Current Protocols in Molecular Biology, ed. Ausubel et al., Greene Publishing and Wiley-Interscience, New York, 1992 (with periodic updates); and Short Protocols in Molecular Biology, ed. Ausubel et al., 52 ed., Wiley-Interscience, New York, 2002. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

FACS analyses are illustratively described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

It is appreciated that all reagents are obtainable by sources known in the art unless otherwise specified. Methods of nucleotide amplification, cell transfection, and protein expression and purification are similarly within the level of skill in the art.

### REFERENCE LIST

Stone, J. H. Limited versus severe Wegener's granulomatosis: baseline data on patients in the Wegener's granulomatosis etanercept trial. Arthritis Rheum 48, 2299-309 (2003).
Reinhold-Keller, E. et al. An interdisciplinary approach to the care of patients with Wegener's granulomatosis: long-term outcome in 155 patients. Arthritis Rheum 43, 1021-32 (2000).
van Rossum, A. P., Limburg, P. C. & Kallenberg, C. G. Activation, apoptosis, and clearance of neutrophils in Wegener's granulomatosis. Ann N Y Acad Sci 1051, 1-11 (2005).
Westman, K. W., Selga, D., Isberg, P. E., Bladstrom, A. & Olsson, H. High proteinase 3-anti-neutrophil cytoplasmic antibody (ANCA) level measured by the capture enzymelinked immunosorbent assay method is associated with decreased patient survival in ANCA-associated vasculitis with renal involvement. J Am Soc Nephrol 14, 2926-33 (2003).
Ayoub, N. et al. Antineutrophil cytoplasmic antibodies of IgA class in neutrophilic dermatoses with emphasis on erythema elevatum diutinum. Arch Dermatol 140, 931-6 (2004).
Ozaltin, F. et al. The significance of IgA class of antineutrophil cytoplasmic antibodies (ANCA) in childhood Henoch-Schonlein purpura. Clin Rheumatol 23, 426-9 (2004).
Jennings, J. G., Chang, L. & Savige, J. A. Anti-proteinase 3 antibodies, their characterization and disease associations. Clin Exp Immunol 95, 251-6 (1994).
Baltaro, R. J. et al. Immunoglobulin G antineutrophil cytoplasmic antibodies are produced in the respiratory tract of patients with Wegener's granulomatosis. Am Rev Respir Dis 143, 275-8 (1991).
Finkielman, J. D. et al. Antiproteinase 3 antineutrophil cytoplasmic antibodies and disease activity in Wegener granulomatosis. Ann Intern Med 147, 611-9 (2007).
Wu, J. et al. FcalphaRI (CD89) alleles determine the proinflammatory potential of serum IgA. J Immunol 178, 3973-82 (2007).
Kocher, M., Edberg, J. C., Fleit, H. B. & Kimberly, R. P. Antineutrophil cytoplasmic antibodies preferentially engage Fc gammaRIIIb on human neutrophils. J Immunol 161, 6909-14 (1998).
Salmon, J. E., Edberg, J. C. & Kimberly, R. P. Fc gamma receptor III on human neutrophils. Allelic variants have functionally distinct capacities. J Clin Invest 85, 1287-95 (1990).
Fanciulli, M. et al. FCGR3B copy number variation is associated with susceptibility to systemic, but not organ-specific, autoimmunity. Nat Genet 39, 721-3 (2007).
Niederer, H. A. et al. Copy number, linkage disequilibrium and disease association in the FCGR locus. Hum Mol Genet 19, 3282-94.
Kessenbrock, K. et al. Netting neutrophils in autoimmune small-vessel vasculitis. Nat Med 15, 623-5 (2009).
Hakkim, A. et al. Impairment of neutrophil extracellular trap degradation is associated with lupus nephritis. Proc Natl Acad Sci USA 107, 9813-8.
Pasquier, B. et al. Identification of FcalphaRI as an inhibitory receptor that controls inflammation: dual role of FcRgamma ITAM. Immunity 22, 31-42 (2005).
Ivashkiv, L. B. Cross-regulation of signaling by ITAM-associated receptors. Nat Immunol 10, 340-7 (2009).
McKinney, E. F. et al. A CD8(+) T cell transcription signature predicts prognosis in autoimmune disease. Nat Med.
Tanaka, S., Edberg, J. C., Chatham, W., Fassina, G. & Kimberly, R. P. Fc gamma RIIIb allele-sensitive release of alpha-defensins: anti-neutrophil cytoplasmic antibody-induced release of chemotaxins. J Immunol 171, 6090-6 (2003).
Kelley J M, Edberg J C, Kimberly R P, Wegener's granulomatosis: a model of auto-antibodies in mucosal autoimmunity, Clin Immunol. 2010 February; 134(2):104-12.
Qian K, Xie F, Gibson A W, Edberg J C, Kimberly R P, Wu J., Functional expression of IgA receptor FcalphaRI on human platelets, J Leukoc Biol. 2008 December; 84(6):1492-500.

Patents and publications mentioned in the specification are indicative of the levels of those skilled in the art to which the invention pertains. These patents and publications are incorporated herein by reference to the same extent as if each individual application or publication was specifically and individually incorporated herein by reference for the material for which they are cited as well as all other material taught or cited therein.

The foregoing description is illustrative of particular embodiments of the invention, but is not meant to be a limitation upon the practice thereof. The following claims, including all equivalents thereof, are intended to define the scope of the invention.

1. A process of treating an autoimmune abnormality in a subject comprising:
determining the sequence of a polymorphic site in a FCAR gene of said subject; and
administering a therapeutic to said subject wherein said therapeutic is selected based on the identity of the FCAR A/G 844 allele.

2. The process of claim 1 further comprising assaying a sample from said subject for the presence or absence IgA anti-neutrophil cytoplasmic antibodies to produce an IgA index.

3. The process of claim 1 further comprising assaying a sample from said subject for the presence or absence of IgG anti-neutrophil cytoplasmic antibodies to produce an IgG index.

4. The process of claim 1 further comprising quantifying said IgA anti-neutrophil cytoplasmic antibodies, quantifying said IgG anti-neutrophil cytoplasmic antibodies, or both.

5. The process of claim 1 further comprising selecting said therapeutic also on the basis of said IgA index, IgG index, or both.

6. The process of claim 1 wherein said therapeutic is exogenous IgA or exogenous IgG.

7. The process of claim 6 further comprising:
assaying a sample from said subject for the presence of endogenous IgA.

8. The process of claim 1 wherein both copies of the FCAR gene of said subject have an A at position 844.

9. The process of claim 8 wherein said therapeutic stimulates CD89 signaling.

10. The process of claim 1 wherein said therapeutic is an inhibitor of CD89 engagement or signaling.

11. The process of claim 10 wherein said inhibitor inhibits downstream signaling of CD89, CD89 engagement, or CD89 ligand binding.

12. The process of claim 1 wherein a FCAR gene of said subject has a G at position 844 and said inhibitor inhibits CD89 engagement or CD89 ligand binding.

13. The process of claim 1 wherein said autoimmune abnormality is Wegener's granulomatosis.

14. A process of screening for a therapeutic to treat an autoimmune abnormality comprising:
contacting a potential therapeutic with a cell associated with an autoimmune abnormality; and
identifying increased, decreased, or unchanged CD89 affector induced activity in said cell.

15. The process of claim 14 wherein said cell is a polymorphonuclear cell.

16. The process of claim 14 wherein said cell is a neutrophil.

17. The process of claim 14 wherein said affector is an activator of CD89 receptor mediated signaling.

18. The process of claim 14 wherein said cell expresses CD89 with an amino acid sequence that includes Gly²⁴⁸.

19. The process of claim 18 wherein said potential therapeutic inhibits downstream signaling of CD89, CD89 engagement, or CD89 ligand binding.

20. The process of claim 14 wherein said cell expresses CD89 with an amino acid sequence that includes Ser²⁴⁸.

21. The process of claim 20 wherein said potential therapeutic engages signaling through CD89.

22. (canceled)

23. A process of diagnosing an autoimmune abnormality in a subject comprising:
assaying a sample from said subject for the presence or absence IgA anti-neutrophil cytoplasmic antibodies to produce an IgA index, and
diagnosing the presence or absence of autoimmune abnormality on the basis of the presence or absence of said antibodies in said sample.

24. The process of claim 23 further comprising: quantifying the IgA anti-neutrophil cytoplasmic antibodies in said sample.

25. The process of claim 23 further comprising:
determining an autoimmune abnormality classification system from a plurality of subjects of known autoimmune condition; and
diagnosing the presence of autoimmune abnormality from comparing said index to said system.

26. The process of claim 23 further comprising:
determining the sequence of a polymorphic site in a FCAR gene of said subject.

27. The process of claim 26 further comprising comparing said sequence to a FCAR sequence from a second subject.

28. The process of claim 23 further comprising:
assaying said sample for the presence or absence of IgG anti-neutrophil cytoplasmic antibodies to produce an IgG index.

29. The process of claim 28 further comprising quantifying said IgG anti-neutrophil cytoplasmic antibodies.

30. The process of claim 23 further comprising predicting the severity of said autoimmune abnormality from a positive IgA index, IgG index, or both.

31. The process of claim 23 further comprising predicting the propensity of renal involvement of said autoimmune abnormality.

32. The process of claim 23 wherein said autoimmune abnormality is Wegener's granulomatosis.

33. (canceled)

34. (canceled)

35. (canceled)

36. (canceled)

37. (canceled)

38. (canceled)

39. (canceled)

40. (canceled)

41. A method for determining a degree of responsiveness subject having an autoimmune abnormality will have to a therapeutic comprising:
ascertaining whether one or more copies of the FCAR gene of said subject has an A or G allele at position 844;
ascertaining whether said subject has a NA1 or NA2 allele of FGCR3B; and
determining the severity of an autoimmune abnormality in a subject on the basis of said FCAR allele, said FGCR3B allele, or both.

42. The process of claim 41 further comprising assaying a sample from said subject for IgA anti-neutrophil cytoplasmic antibodies to produce an IgA index, and determining the severity of an autoimmune abnormality also on the basis of the presence or absence of said antibodies in said sample.

43. The process of claim 41 further comprising determining the autoimmune abnormality classification category of said subject based on comparing said FCAR allele and said FGCR3B allele to a classification system wherein said determining is based on disease severity, disease activity, or propensity of disease from subjects with matching classification category.

44. The process of claim 43 further comprising assaying a sample from said subject for the presence or absence of IgA anti-neutrophil cytoplasmic antibodies to produce an IgA index, IgG anti-neutrophil cytoplasmic antibodies to produce an IgG index, or both.

45. The process of claim 44 further comprising determining the autoimmune abnormality classification category of said subject based on comparing said IgA index, said FCAR allele, and said FGCR3B allele to a classification system wherein said determining is based on disease severity, disease activity, or propensity of disease from subjects with matching classification category.

46. The process of claim 41 wherein said therapeutic is an antibody.

47. The process of claim 41 wherein said therapeutic is a plasma-derived immunoglobulin.

48. (canceled)
